(19) United States
(12) Patent Application Publication
(10) Pub. No.: US 2019/0383779 A1
(43) Pub. Date: Dec. 19, 2019

(54) METHOD FOR CONTROLLING THE CONCENTRATION OF VOLATILE ORGANIC COMPOUNDS IN A FLUID OF A FLUID NETWORK

(71) Applicant: SUEZ GROUPE, Paris La Défense Cedex (FR)

(72) Inventors: Auguste BRUCHET, CHATOU (FR); Zdravka DO QUANG, BAILLY (FR); Isabelle BAUDIN, NANTERRE (FR); Naike NOYON, IGNY (FR)

(21) Appl. No.: 16/463,828

(22) PCT Filed: Dec. 5, 2017
(86) PCT No.: PCT/EP2017/081444

§ 371 Date: May 23, 2019

(30) Foreign Application Priority Data
Dec. 8, 2016 (FR) 1662143

(51) Int. Cl. G01N 33/00; G01N 33/18; G01N 33/28

(52) U.S. Cl. CPC G01N 33/0063; G01N 33/2823; G01N 33/0047; G01N 33/1826

(57) ABSTRACT

A process for monitoring the concentration of organic compounds in a fluid of a network, the fluid includes volatile organic compounds, comprising the following steps: first step of on-line measurement of the concentration of each of the organic compounds of the organic compounds in the fluid by an analyzer, second step of comparing the concentration of each of the organic compounds measured by the analyzer with a predefined threshold value for each of the organic compounds, third step of reducing the concentration of at least one organic compound if the concentration of the at least one organic compound measured by the analyzer is greater than the corresponding predefined threshold value. A device for monitoring the concentration of organic compounds is also provided.

The invention lies in the field of the on-line measurement of organic compounds in a fluid network and relates to a process for monitoring the concentration of organic compounds in a fluid of a fluid network. The invention also relates to a device for monitoring the concentration of organic compounds in a fluid of a fluid network. The invention may be applied to the monitoring of any type of fluid, whether this is cooling network waters, natural waters, environmental waters or treated waters, or gaseous fluid such as air. The invention applies to volatile or nonvolatile organic compounds.

Drinking waters are defined by several tens of nonspecific parameters (temperature, pH, conductivity, turbidity, etc.), microbiological parameters (coliforms, total bacteria, etc.) and organic chemical parameters (pesticides, hydrocarbons, chlorinated solvents, trihalomethanes, etc.) and inorganic chemical parameters (metals, bromates, etc.). Compliance is monitored by means of samplings and laboratory analyses, the results of which are known within a period ranging from 12 hours to a few weeks. Furthermore, the frequency of these analyses may vary in the best case scenario from daily (microbiology at large plants) to once a month for the organic and inorganic chemical parameters, this being for large plants. For plants delivering less than 1000 m³/day, this frequency may only be annual. It is therefore clear that in the intervals between these laboratory analyses instances of nonconformity may occur without being detected, therefore without the possibility of action at the treatment plants. An ideal solution would consist in controlling the treatment plants using information provided daily or several times a day, by continuous or semi-continuous measurement systems, upstream and/or downstream of the treatments.

Continuous measurement probes already exist for a limited number of simple physicochemical parameters such as pH, turbidity, conductivity, temperature, UV absorbance, but no treatment plant that makes it possible to monitor the concentration of organic compounds has been identified to date.

It therefore appears necessary to have a treatment plant controlled or optimized by information originating from on-line analyses of specific (individual) organic compounds in particular of volatile organic compounds (VOCs). VOCs being defined in France (decree no. 2006-623) as any organic compound with a boiling point, measured at the standard pressure of 101.3 kPa, of less than or equal to 250° C. (cf. table below). VOCs should be considered owing to their toxicity or generation of tastes and odors. The compounds particularly relevant for drinking water applications are those that occur most in resources, some of which are regulated (in bold in the table below): vinyl chloride, 1,1-dichloroethane, ETBE, 1,1,1-trichloroethane, chloroform, cis-1,2-dichloroethylene, trichloroethylene and tetrachloroethylene:

| | | Saturated vapor | |
|---|---|---|---|
| | | pressure | Boiling |
| Family | Compound | (Pa at 20° C.) | point |
| Halogenated | dichloromethane | 4.65 × 10⁴ | 40° C. |
| hydrocarbons | trichloroethylene | 8.6 × 10³ | 87° C. |
| (VHOC) | tetrachloroethylene | 1.9 × 10³ | 121° C. |
| Aromatic | benzene | 9.97 × 10³ | 80.1° C. |
| hydrocarbons | toluene | 3.9 × 10³ | 110.58° C. |
| (BTEX) | o-xylene | 663 | 144.43° C. |
| | ethylbenzene | 900 | 136.2° C. |
| Ethers | methyl tert-butyl ether | 2.7 × 10⁴ | 55.05° C. |
| | (MTBE) | | |
| | ethyl tert-butyl ether | 1.7 × 10⁴ | 73° C. |
| | (ETBE) | | |

| | | France | | United States |
|---|---|---|---|---|
| Parameter | WHO (2011) | (directive 98/83/CE) | Australia | (US EPA) |
| 1,1-dichloroethylene | | | | 7 μg/L |
| 1,2-dichloroethylene | 50 μg/L | | | |
| cis-1,2-dichloroethylene | | | | 70 μg/L |
| trans-1,2-dichloroethylene | | | | 100 μg/L |
| 1,2-dichloroethane | 30 μg/L | 3 μg/L | 3 μg/L | 5 μg/L |
| 1,2-dichlorobenzene | 1 000 μg/L | | | |
| 1,1,1-trichloroethane | | | | 200 μg/L |
| 1,2,4-trichlorobenzene | | | | 70 μg/L |
| Benzene | 10 μg/L | 1 μg/L | 1 μg/L | 5 μg/L |
| Chlorobenzenes | | | | 100 μg/L |
| Chloroform | 300 μg/L | ΣTHMs = 100 μg/L | | 80 μg/L |
| Vinyl chloride | 0.3 μg/L | 0.5 μg/L | 0.3 μg/L | 2 μg/L |
| Dichloromethane | 20 μg/L | | | 5 μg/L |
| Ethylbenzene | 300 μg/L | | | 700 μg/L |
| Styrene | 20 μg/L | | | 100 μg/L |
| Carbon tetrachloride | 4 μg/L | | | 5 μg/L |
| Tetrachloroethylene | 40 μg/L | Σ = 10 μg/L | | 5 μg/L |
| Trichloroethylene | 20 μg/L | | | 5 μg/L |
| Toluene | 700 μg/L | | | 1 000 μg/L |
| Xylenes (total) | 500 μg/L | | | 10 μg/L |

But there is a prejudice linked to the complexity of the laboratory chromatographs and to the expertise required in order to use them.

The invention aims to overcome all or some of the problems cited above by providing a process for monitoring the concentration of organic compounds in a fluid of a fluid network by carrying out an on-line measurement of the organic compounds and the result of the measurements of which makes it possible to control an industrial process so as to reduce the concentration of organic compounds if this concentration is too high. This process makes it possible to improve the operational use of the data originating from the monitoring of the organic compounds for the water networks. It makes it possible to guarantee performances identical to those of laboratory reference methods (with a relatively low maximum allowable deviation) and therefore to optimize the operational parameters of the treatment line without risk of overmetering or undermetering of products intended to reduce the concentration of the organic compounds.

For this purpose, one subject of the invention is a process for monitoring the concentration of organic compounds in a fluid of a fluid network of an industrial plant, the fluid comprising a plurality of organic compounds, characterized in that it comprises the following steps:

first step of on-line measurement of the concentration of each of the organic compounds of the plurality of organic compounds in the fluid of the fluid network by an analyzer,

second step of comparing the concentration of each of the organic compounds of the plurality of organic compounds measured by the analyzer with a predefined threshold value for each of the organic compounds, such as treatment setpoint value or maximum alarm value,

third step of reducing the concentration of at least one organic compound if the concentration of said at least one organic compound measured by the analyzer is greater than the corresponding predefined threshold value.

According to one embodiment, the reducing third step comprises a step of injecting into the fluid network a product that has the effect of reducing the concentration of at least one organic compound. It may be a step of injecting into the fluid network a reactant (such as an adsorbent media, an oxidizer, a stripping gas) or use of a treatment process (such as membrane filtration).

According to one embodiment, the industrial plant is intended to carry out an industrial process, and the reducing third step consists in acting on at least one parameter of the industrial process having the effect of reducing the concentration of said at least one organic compound.

According to another embodiment, the monitoring process according to the invention comprises, prior to the reducing third step, a step of remote transmission of the concentration of each of the organic compounds of the plurality of organic compounds measured by the analyzer to a control center.

According to one embodiment, the industrial process is a process for treating a liquid fluid by aeration using a gaseous fluid in a predefined ratio between the liquid fluid and the gaseous fluid, and the reducing third step consists in adjusting the ratio between the liquid fluid and the gaseous fluid as a function of the concentration of the at least one organic compound measured in the fluid of the network.

According to another embodiment, the industrial process is a drilling process using a drilling fluid, and the reducing third step consists in adapting the density and/or the composition of the drilling fluid as a function of the concentration of the at least one organic compound measured in the fluid of the network.

According to another embodiment, the industrial process is a process for disinfecting water by addition of at least one oxidizing reactant, and the reducing third step consists in adapting the amount of at least one reactant (such as chlorine) added as a function of the concentration of the at least one organic compound measured in the fluid of the network.

According to another embodiment, the industrial process is a process for treating drinking water, the industrial plant comprising at least one filter with granular adsorbent media such as activated carbon, or an injection of powdered media, such as activated carbon, and the reducing third step comprises a step of varying the flow rate of the fluid through an adsorbent/fluid contactor, such as filter with granular adsorbent media, or powdered adsorbent media/fluid separator. The metering of the powdered adsorbent media may also be adapted to the desired abatement of the organic compound(s) and to the on-line monitoring.

Advantageously, the reducing third step may further comprise a step of regenerating the filter media and the regeneration of the media is carried out at a fixed or variable frequency.

The industrial treatment process may also be a process based on hydrophilic membranes permeable to liquid fluids or based on hydrophobic membranes permeable to gaseous fluids, of nanofiltration or reverse osmosis type.

The invention also relates to a device for monitoring the concentration of organic compounds in a fluid of a fluid network of an industrial plant suitable for carrying out an industrial process, the fluid comprising a plurality of organic compounds, the device comprising an analyzer positioned at a first location in the fluid network, suitable for on-line measuring of the concentration of each of the organic compounds of the plurality of organic compounds in the fluid at the first location, a comparator suitable for comparing the concentration of each of the organic compounds of the plurality of organic compounds measured by the analyzer with a predefined threshold value for each of the organic compounds, a correction unit suitable for reducing the concentration of at least one organic compound in the fluid, if the concentration of said at least one organic compound of the plurality of organic compounds measured by the analyzer is greater than the corresponding predefined threshold value.

Advantageously, the correction unit comprises a device for injecting into the fluid network a product that has the effect of reducing the concentration of said at least one organic compound.

According to one embodiment, since the industrial process involves at least one parameter, the correction unit comprises a first control module configured to act on the at least one parameter of the industrial process having the effect of reducing the concentration of said at least one organic compound as a function of the change in concentration of said at least one organic compound measured.

According to another embodiment, the monitoring device comprises a remote transmission device suitable for remotely transmitting the concentration of each of the organic compounds of the plurality of organic compounds measured by the analyzer to a control center.

The invention will be better understood and other advantages will become apparent on reading the detailed description of an embodiment given by way of example, which description is illustrated by the appended drawing wherein:

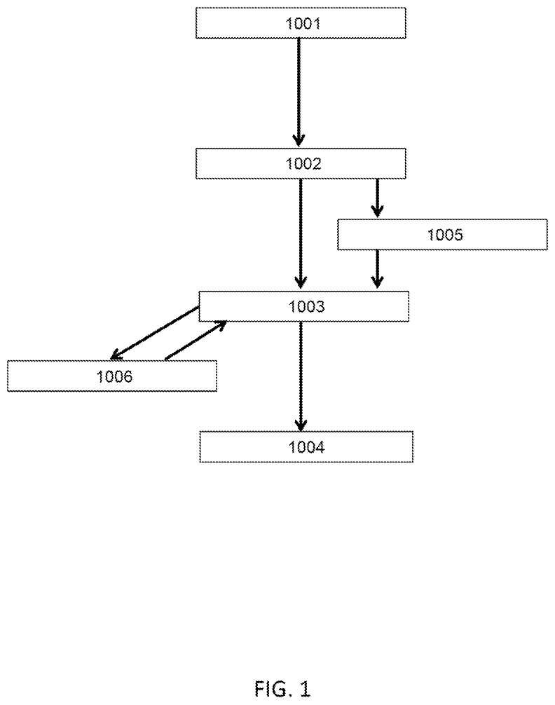

FIG. 1 schematically represents the steps of a process for monitoring the concentration of organic compounds in a fluid of a network according to the invention,

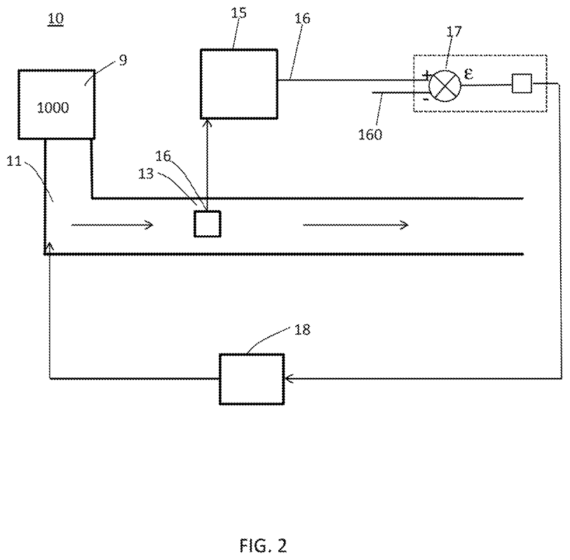

FIG. 2 schematically represents an embodiment of a device for monitoring the concentration of organic compounds in a fluid of a network according to the invention,

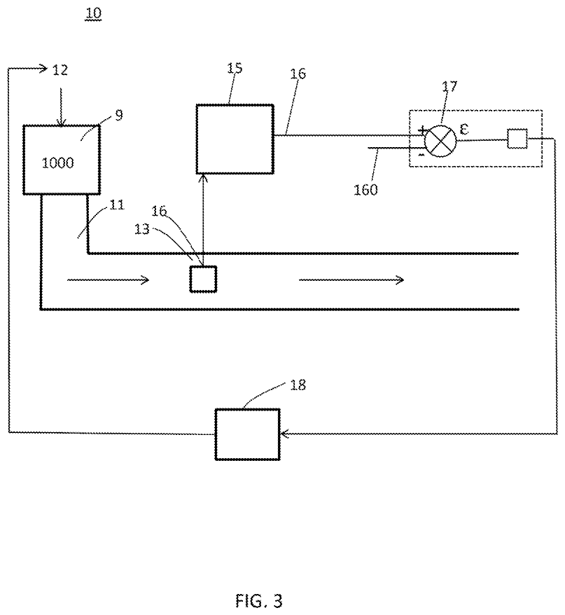

FIG. 3 schematically represents another embodiment of a device for monitoring the concentration of organic compounds in a fluid of a network according to the invention.

For the sake of clarity, the same elements will bear the same references in the various figures.

FIG. 1, while also referring to FIG. 2, schematically represents the steps of a process for monitoring the concentration of organic compounds in a fluid 11 of a network according to the invention. The process for monitoring the concentration of organic compounds in the fluid 11 of the fluid network of an industrial plant, the fluid 11 comprising a plurality of organic compounds, comprises a first step 1001 of on-line measurement of the concentration 16 of each of the organic compounds of the plurality of organic compounds in the fluid 11 of the fluid network by an analyzer 15, a second step 1002 of comparing the concentration 16 of each of the organic compounds of the plurality of organic compounds measured by the analyzer 15 with a predefined threshold value 160 for each of the organic compounds, such as treatment setpoint value or maximum alarm value, and a third step 1003 of reducing the concentration 16 of at least one organic compound if the concentration 16 of said at least one organic compound measured by the analyzer 15 is greater than the corresponding predefined threshold value 160.

The analyzer 15 is configured for the on-line measurement of the concentration 16 of each of the organic compounds of the plurality of organic compounds present in the fluid 11. This results in a plurality of concentrations 16, one for each organic compound measured. And for each of the organic compounds, a threshold value 160 is predefined, each of the values possibly being different depending on the standards in force. Each concentration of organic compound measured is compared to the threshold value corresponding to the organic compound in question. If the concentration of organic compound measured is greater than this threshold value, a corrective action is required corresponding to the third step 1003 for reducing the concentration of this organic compound.

The reducing third step 1003 may in particular comprise a step 1004 of injecting into the network of fluid 11 a product that has the effect of reducing the concentration of the at least one organic compound. The injection of the product may be subjected to a closed-loop control that is controlled by the difference between the measured concentration and the predefined threshold value of the concentration of the organic compound.

The monitoring process according to the invention may comprise, prior to the reducing third step 1003, a step 1005 of remote transmission of the concentration 16 of each of the organic compounds of the plurality of organic compounds measured by the analyzer 15 to a control center of the treatment plant. The control center may then also act on the concentration of the organic compounds, optionally via a human intervention.

One particularly advantageous aspect of the invention applies to an industrial plant 9 intended to carry out an industrial process 1000. According to the invention, the reducing third step 1003 consists in acting on at least one parameter 12 of the industrial process 1000 having the effect of reducing the concentration 16 of said at least one organic compound having a concentration measured by the analyzer 15 greater than its predefined threshold value 160.

The invention therefore makes it possible, from the on-line measurement of the concentration of organic compounds, to control and optimize (in terms of performance and operating costs) industrial processes in the world of water and the environment. For example, the invention contributes to the prevention of industrial pollution by chlorinated solvents in water resources to improve the treatment system. By controlling operational parameters on the industrial plant upstream, the invention makes it possible to monitor byproducts of chlorine disinfection, such as trihalomethanes which are volatile organic compounds, and which are the subject of recommendations by the World Health Organisation or of limit values in the United States, in Europe and in Australia. Within the context of the invention, it is also possible to identify and treat air pollutions produced for example by the wastewater industry and the treatment of sludges which generate olfactory pollutants.

According to one embodiment, the industrial process 1000 is a process for treating a liquid fluid by aeration using a gaseous fluid in a predefined ratio between the liquid fluid and the gaseous fluid, and the reducing third step 1003 consists in adjusting the ratio between the liquid fluid and the gaseous fluid as a function of the concentration of the at least one organic compound measured in the fluid of the network. This results in a good monitoring of the efficiency of the treatment process, also referred to as a stripping process, and in optimizing the operating costs thereof.

According to another embodiment, the industrial process is a drilling process using a drilling fluid, and the reducing third step 1003 consists in adapting the density and/or the composition of the drilling fluid as a function of the concentration of the at least one organic compound measured in the fluid of the network. In other words, the invention enables a good monitoring of contaminated drillings with control of the mixture of contaminated/uncontaminated drillings, so as to comply with the legislation regarding distributed water.

According to another embodiment, the industrial process is a process for disinfecting water by addition of at least one reactant, and the reducing third step 1003 consists in adapting the amount of the at least one reactant added as a function of the concentration of the at least one organic compound measured in the fluid of the network.

According to another embodiment, the industrial process is a process for treating drinking water, the industrial plant comprising at least one filter with granular adsorbent media, such as granular activated carbon, or else an injection of powdered adsorbent media or a contactor with activated carbon powdered adsorbent media or a membrane separation process, and the reducing third step 1003 comprises a step of varying the flow rate of the fluid through the adsorbent/fluid contactor, such as the filter with granular adsorbent material or media, or powdered adsorbent media/fluid separator or in the contactor with powdered adsorbent media. The third step 1003 may also comprise a step of varying the metering of the powdered adsorbent media. That is to say that the metering of the powdered adsorbent media may also be adapted to the desired abatement of the organic compound(s) and to the on-line monitoring.

In other words, a filter is located in the treatment plant. A drinking water treatment plant often comprises activated carbon filters intended to eliminate micro-pollutants. By acting on the flow rate of water through such filters and on their frequency of regeneration, it is possible to improve the elimination of the organic compounds. Use may also be made, in the plant, of a reactor for contact of water/powdered adsorbent media, such as powdered activated carbon, which are intended to eliminate micro-pollutants. By acting on the flow rate of water through such a contactor and on the metering of the powdered media, it is possible to improve the elimination of the organic compounds.

Advantageously, the reducing third step 1003 further comprises a step 1006 of regenerating the filter media and the regeneration of the media is carried out at a fixed or variable frequency.

The industrial process 1000 may also be a process for treating drinking water, the industrial plant comprising at least one hydrophilic membrane permeable to liquid fluids or at least one hydrophobic membrane permeable to gaseous fluids, of nanofiltration or reverse osmosis type. And in this case, the reducing third step 1003 comprises a step of varying the flow rate of the fluid across the membrane.

Following the measurement carried out by the analyzer 15, in the event of exceeding the regulatory value, the result of the measurement carried out on-line by the analyzer 15 is used to feedback to the operation of the industrial treatment plant by lowering for example the dose of chlorine or by optimizing the elimination of the organic materials by acting on the amount of coagulant or the dose of powdered activated carbon, or on the frequency of regeneration of the carbon filters.

The implementation of the process according to the invention makes it possible to have a measurement of the concentration of organic compounds in a relevant fluid, since it is carried out on-line and continuously or semi-continuously, and a useful result for improving the industrial process associated with the fluid.

The industrial process is controlled in real time, which makes it possible to be more reactive faced with any possible industrial pollution. This real-time control is not conceivable in the case of fluid sampling for analysis of the concentrations of organic compounds in a laboratory, because the turnaround time is too long. Furthermore, since the organic compounds may be volatile organic compounds, with a laboratory analysis there is a problem of accuracy of the measurement as the volatile organic compound has high chances of no longer being in the sample analyzed. Therefore, the control of the industrial process according to the invention makes it possible to be more relevant and closer to reality by actually capturing the pollution and by treating it in an appropriate manner. The resulting advantage is a saving in time, cost and quality of the treatment of the fluid.

FIG. 2 schematically represents an embodiment of a device 10 for monitoring the concentration 16 of organic compounds in a fluid 11 of a network according to the invention. The device 10 for monitoring the concentration 16 of organic compounds in the fluid 11 of the network of fluid 11 of an industrial plant 9 suitable for carrying out an industrial process 1000, comprises an analyzer 15 positioned at a first location 13 in the fluid network, suitable for on-line measuring of the concentration 16 of each of the organic compounds of the plurality of organic compounds in the fluid 11 at the first location 13. The analyzer 15 is a machine capable of measuring on-line, continuously or semi-continuously, several organic compounds that are regulated or frequently found in the waters or any other related fluid. The monitoring device 10 comprises a comparator 17 suitable for comparing the concentration 16 of each of the organic compounds of the plurality of organic compounds measured by the analyzer 15 with a predefined threshold value 160 for each of the organic compounds, and a correction unit 18 suitable for reducing the concentration 16 of at least one organic compound in the fluid 11, if the concentration of said at least one organic compound of the plurality of organic compounds measured by the analyzer 15 is greater than the corresponding predefined threshold value 160.

The analyzer 15 may for example be a portable gas chromatograph coupled to a micro argon ionization detector, comprising a sampling device. The analyzer 15 may comprise two probes, one a temperature probe and the other enabling the inlet of argon into the fluid. Two types of containers may be fastened thereto. One is used to carry out the static tests and may have a capacity of 2 L. The second container is used for the on-line analyses. It is equipped with two openings, one for the inlet of the fluid and the other for the outlet, which are connected to pipes to form the circuit. Advantageously, the analyzer 15 is started up as soon as it is connected to a power outlet. The data processing is carried out on a computer equipped with suitable software networked with the analyzer 15. It must be connected to an argon cylinder in order to operate. The on-line analyzer is made to operate continuously with minimal maintenance. The analysis may be conducted without pretreatment or prefiltration for waters that are not heavily loaded.

For the analyses of water, the latter circulates continuously through a lower cell. At a programmable frequency, the organic compounds of the circulating water are stripped by an inert gas (in this case argon), conveyed by the argon over an adsorbent which traps them. The latter is then desorbed thermally and the organic compounds are conveyed by the argon carrier gas to a chromatography column which separates them physically. The separated compounds are then detected by means of a micro argon ionization detector that requires argon for the operation thereof. This system therefore operates on the dynamic headspace principle.

It should be noted that this is an example of an analyzer 15 that may be used within the context of the invention. The invention may be applied with any type of analyzer capable of separating the organic compounds individually on-line, continuously or semicontinuously, in order to determine the concentration thereof.

Advantageously, the correction unit 18 may comprise a device for injecting into the network of fluid 11 a product having the effect of reducing the concentration of said at least one organic compound.

According to another embodiment, the monitoring device 10 comprises a remote transmission device suitable for remotely transmitting the concentration 16 of each of the organic compounds of the plurality of organic compounds measured by the analyzer to a control center.

FIG. 3 schematically represents another embodiment of the device 10 for monitoring the concentration of organic compounds in the fluid 11 of the network according to the invention. The industrial process 1000 involves at least one parameter 12, the correction unit 18 comprises a first control module configured to act on the at least one parameter 12 of the industrial process 1000 having the effect of reducing the concentration of said at least one organic compound as a function of the change in concentration of said at least one organic compound measured.

Such a monitoring device 10 according to the invention therefore makes it possible to obtain an on-line measurement of the concentration of organic compounds in a fluid, and to control the industrial process associated with the fluid. More specifically, such a monitoring device makes it possible to identify and treat pollutions by acting on an operating parameter of the industrial process in order to best adapt the operation of the industrial plant while taking into account the level of the organic compounds that result therefrom. As already mentioned, the organic compounds may be chlorinated solvents in water, trihalomethanes, but also additives in particular dedicated to fuels such as ethyl tert-butyl ether (ETBE), or methyl tert-butyl ether (MTBE), or else even odorous algal metabolites on condition that the detection threshold of the analyzer is low enough. Of course, the invention applies to any volatile organic compound.

1. A process for monitoring the concentration of volatile organic compounds in a fluid of a fluid network of an industrial plant, the fluid comprising a plurality of volatile organic compounds, wherein it comprises the following steps:
first step of on-line measurement of the concentration of each of the volatile organic compounds of the plurality of volatile organic compounds in the fluid of the fluid network by an analyzer,
second step of comparing the concentration of each of the volatile organic compounds of the plurality of organic compounds measured by the analyzer with a predefined threshold value for each of the volatile organic compounds, such as treatment setpoint value or maximum alarm value, third step of reducing the concentration of at least one volatile organic compound if the concentration of said at least one volatile organic compound measured by the analyzer is greater than the corresponding predefined threshold value.

2. The monitoring process as claimed in claim 1, wherein the reducing third step comprises a step of injecting into the fluid network a product that has the effect of reducing the concentration of at least one volatile organic compound.

3. The monitoring process as claimed in claim 1, the industrial plant being intended to carry out an industrial process, wherein the reducing third step consists in acting on at least one parameter of the industrial process having the effect of reducing the concentration of at least one volatile organic compound.

4. The monitoring process as claimed in claim 1, wherein any one of the preceding claims, characterized in that it comprises, prior to the reducing third step, a step of remote transmission of the concentration of each of the volatile organic compounds of the plurality of volatile organic compounds measured by the analyzer to a control center.

5. The monitoring process as claimed in claim 3, wherein
the industrial process is a process for treating a liquid fluid by aeration using a gaseous fluid in a predefined ratio between the liquid fluid and the gaseous fluid,
and in that the reducing third step consists in adjusting the ratio between the liquid fluid and the gaseous fluid as a function of the concentration of the at least one volatile organic compound measured in the fluid of the network.

6. The monitoring process as claimed in claim 3, wherein
the industrial process is a drilling process using a drilling fluid, and in that the reducing third step consists in adapting the density and/or the composition of the drilling fluid as a function of the concentration of the at least one volatile organic compound measured in the fluid of the network.

7. The monitoring process as claimed in claim 3, wherein
the industrial process is a process for disinfecting water by addition of at least one reactant,
and in that the reducing third step consists in adapting the amount of the at least one reactant added as a function of the concentration of the at least one volatile organic compound measured in the fluid of the network.

8. The monitoring process as claimed in claim 3, wherein
the industrial process is a process for treating drinking water, the industrial plant comprising at least one filter with granular adsorbent media, or a contactor with activated carbon powdered adsorbent media or a membrane separation process,
and in that the reducing third step comprises a step of varying the flow rate of the fluid through the filter with granular adsorbent media, or in the contactor with powdered adsorbent media, or a step of varying the metering of the powdered adsorbent media.

9. The monitoring process as claimed in claim 8, wherein the reducing third step further comprises a step of regenerating the filter media and in that the regeneration of the filter media is carried out at a fixed or variable frequency.

10. The monitoring process as claimed in claim 3, wherein
the industrial process is a process for treating drinking water, the industrial plant comprising at least one hydrophilic membrane permeable to liquid fluids or at least one hydrophobic membrane permeable to gaseous fluids,
and in that the reducing third step comprises a step of varying the flow rate of the fluid across the membrane.

11. A device for monitoring the concentration of volatile organic compounds in a fluid of a fluid network of an industrial plant suitable for carrying out an industrial process the fluid comprising a plurality of volatile organic compounds, wherein it comprises:
an analyzer positioned at a first location in the fluid network, suitable for on-line measuring of the concentration of each of the volatile organic compounds of the plurality of volatile organic compounds in the fluid at the first location,
a comparator suitable for comparing the concentration of each of the volatile organic compounds of the plurality of volatile organic compounds measured by the analyzer with a predefined threshold value for each of the volatile organic compounds,
a correction unit suitable for reducing the concentration of at least one volatile organic compound in the fluid, if the concentration of said at least one volatile organic compound of the plurality of organic compounds measured by the analyzer is greater than the corresponding predefined threshold value.

12. The monitoring device as claimed in claim 11, wherein the correction unit comprises a device for injecting into the fluid network a product that has the effect of reducing the concentration of said at least one volatile organic compound.

13. The monitoring device as claimed in claim 11, wherein the correction unit comprises a first control module configured to act on at least one parameter of the industrial process having the effect of reducing the concentration of said at least one volatile organic compound as a function of the change in concentration of said at least one volatile organic compound measured.

14. The monitoring device as claimed in claim 11, wherein it comprises a remote transmission device suitable for remotely transmitting the concentration of each of the volatile organic compounds of the plurality of volatile organic compounds measured by the analyzer to a control center.
